(19) United States
(12) Patent Application Publication
(10) Pub. No.: US 2018/0179357 A1
(43) Pub. Date: Jun. 28, 2018

(54) ENERGY DISSIPATIVE COMPOSITION INCLUDING A HYDROGEL REINFORCED WITH NANOPOROUS PARTICLES

(71) Applicant: Board of Trustees of Michigan State University, East Lansing, MI (US)

(72) Inventors: Weiyi Lu, Ann Arbor, MI (US); Roozbeh Dargazany, East Lansing, MI (US)

(73) Assignee: Board of Trustees of Michigan State University, East Lansing, MI (US)

(21) Appl. No.: 15/839,917

(22) Filed: Dec. 13, 2017

Related U.S. Application Data
(60) Provisional application No. 62/439,240, filed on Dec. 27, 2016.

(51) Int. Cl. C08J 9/00; C08K 7/26; C08K 9/06; A61L 27/52; A61L 27/56; A61L 27/44; A61L 27/26; A61L 31/14; A61L 31/12; A61L 31/04

(52) U.S. Cl. CPC C08J 9/008; C08J 2205/022; C08K 9/06; A61L 27/52; A61L 27/56; A61L 27/44; A61L 27/26; A61L 31/145; A61L 31/146; A61L 31/125; A61L 31/041; A61L 2430/02; A61L 2430/24; A61L 2430/06; C08K 2201/011; C08J 2333/02; C08K 7/26

(57) ABSTRACT

A composition includes hydrogel and nanoporous particles having an internal cavity without any liquid therein in an ambient condition. In another aspect, a hybrid hydrogel includes particles having vacant or liquid-free internal cavities in a first condition and allowing entry of a liquid in a second condition, to absorb impact energy. A further aspect employs particle pores into which hydrogel liquid flows when impacted. Moreover, another aspect of the present hydrogel and nanoporous particle composite is used in biomedical inserts, stretchable biometric sensors, vehicular armor, wearable helmets or armored garments, or padded vehicular interior components such as seats, headrests, instrument panels or door trim panels.

### CROSS-REFERENCE TO RELATED APPLICATIONS

This application claims the benefit of U.S. Provisional Application No. 62/439,240, filed on Dec. 27, 2016. The entire disclosure of the above application is incorporated by reference herein.

### BACKGROUND AND SUMMARY

The present disclosure generally pertains to hydrogels and more particularly to hydrogels including nanoporous particles.

A hydrogel is a network of molecular chains into which a liquid is absorbed or trapped, forming a material that is typically 80 percent or more water. Attempts have been made to use hydrogels as bio-materials for the replacement of ligaments, tendons and other biological tissues, because of their high water content and low friction coefficient. In most applications, the hydrogel composites can be easily stretched to a large elastic while having a wide range of stiffnesses. Unfortunately, conventional single-network (“SN”) hydrogels are brittle and can be fractured at extremely low stresses even under compression. This poor mechanical behavior limits their use as load carrying components. The low toughness comes from irreversible permanent damage in the hydrogels, such as sliding or scission of the polymer network chains.

One conventional hydrogel and nanoparticle composition is disclosed in U.S. Patent Publication No. 2016/0303281 entitled “Composition and Kits for Pseudoplastic Microgel Matrices” which published to Salamone et al. on Oct. 20, 2015, and is incorporated by reference herein. This composition can be injected for use as a scaffold matrix. The nanoparticles in this conventional composition, however, are pre-filled with a medically or biologically active agent in an ambient condition. Furthermore, the nanoparticles are hydrophilic.

In an effort to improve the mechanical properties of traditional SN hydrogels, various types of additives have been employed to reinforce their microstructure including nanocomposites, fibers, copolymers, and multiple networks. The failure strength and toughness of hydrogels can be improved by selecting stronger matrix materials, adding a copolymer, and increasing fiber content or crosslink density. However, these traditional additives have several significant drawbacks:
Reduced flexibility of gels—The additives significantly increase the stiffness of the reinforced hydrogels. In general, the failure strength and fracture toughness of the hydrogel cannot be improved at the same time. Thus, despite theoretically achieving a greater strength of the reinforced hydrogels, they may fracture at even lower energy levels because of reduced ductility.
Hard-to-control constitutive behavior—There is no direct relationship between the compound and the behavior of the gels. Usually the relationships are explored by expensive trial and error compounding.
Rough surface of the gels—The surface properties of additives and the increased stiffness decrease the swelling ratio of hydrogel composites and reduce water content. As a result, the coefficient of friction increases thereby leading the reinforced hydrogels to have unfavorable bio-compatibility.

Furthermore, various attempts have been made to add conventional silica nanoparticles or microparticles in hydrogels for drug delivery. Such a composite is disclosed in U.S. Patent Publication No. 2016/0136088 entitled “Silica Hydrogel Composite” which published to Jokinen et al. on May 19, 2016, and is incorporated by reference herein. These hydrogel composites, however, are injectable into a patient's tissue and the pharmaceutical ingredients are encapsulated within the particles in an ambient condition prior to the injection.

Experimental tests have been conducted on liquid infiltration in nanoparticles. See for example, Surani, F., et al., “Thermal Recoverability of a Polyelectrolyte-Modified, Nanoporous Silica-Based System,” J. Mater. Res., Vol. 21, No. 9 (September 2006), and Surani, F. et al., “Energy Absorption of a Polacrylic Acid Partial Sodium Salt-Modified Nanoporous System,” J. Mater. Res., Vol. 21, No. 5 (May 2006). However, Lu, W., “Experimental Investigation on Liquid Behaviors in Nanopores,” University of California San Diego Electronic Theses and Dissertations (2011) at p. 105, notes that [w]hile hydrogel matrix NMF [liquid] composites have been developed . . . , they can merely stand alone and still cannot be directly used for load-bearing components.”

In accordance with the present invention, a composition includes a hydrogel and nanoporous particles having an internal cavity, such as a pore or void, without any liquid therein in an ambient condition. In another aspect, a hybrid hydrogel includes nanoporous particles having vacant or liquid-free internal cavities, such as nanopores or voids, in a first condition and allowing entry of a liquid in a second condition, to absorb or dissipate impact energy. A further aspect employs nanopores into which hydrogel liquid flows when impacted. Moreover, another aspect of the present hydrogel and nanoporous particle composition is used as a load-bearing component in biomedical inserts, stretchable biometric sensors, vehicular armor, wearable helmets, wearable armored garments, and padded vehicular interior components such as seats, headrests, instrument panels, door trim panels or the like. Methods of making and using a hydrogel with nanoporous particles are also provided.

The present hybrid hydrogel with nanoporous particles is advantageous over conventional compounds and devices. For example, the present use of nanoporous particles (“NpP”) as a reinforcement for hydrogels improves mechanical properties of the hydrogels and also allows for customized programming or tailoring of their response characteristics. This is expected to achieve the following advantages:
Minimizes loss of flexibility—The stiffness of the present nanoporous particles is lower than an equivalent solid particle due to the presence of the internal cavities, such as pores or voids. Therefore, the present hybrid hydrogels are considerably softer than those reinforced by conventional methods.
Added (reserve) toughness—An additional toughness can be provided in the present material through a liquid infiltration process into the nanoporous particle which is a process similar to an implosion of water bubbles. The toughness added to the hydrogels by adding the present nanoporous particle (so-called “reserve toughness”) is independent of an interaction between the porous particles and the polymer chains. These interactions, however, can influence the classical toughness of the composition. This influence can be minimized if nanoporous particles with non-functionalized surfaces are used in the present composition.
Programmability: The added toughness can be programmed to be triggered at specific deformation or stress level. Such programming will take place via changes in the morphology, surface properties, nanopore structure, and load fraction of the NpP, as well as the chemistry of hydrogel network. Such a programmable response allows tailoring of the constitutive behavior by an addition of multiple type of nanoporous particles. Before the activation level, the material will act like conventional hydrogels; however, after activation, the energy absorption capacity of the material increases considerably which prevents the material from failure. The programmability of the gel allows it to be used for efficient impact absorption rather than wave reflection specifically, where a thin film of the gel can be substituted for current elastomer paddings with several inches of thickness prior to ultimate failure.
Enhances failure strength and toughness—The reinforcement function in the present hybrid hydrogel composition is the result of two independent mechanisms: (a) strong interaction between the surface of the nanoporous particles and polymer chains due to extremely large specific surface areas of the nanoporous particles for functionalized surfaces, and (b) an infiltration process at a specific stress level.
Smooth surface of the filled hydrogels—The effect of the nanoporous particle additive on the surface roughness of the hybrid hydrogel is considerably small, in contrast to conventional toughening methods used to process reinforced hydrogels.

Furthermore, the present hybrid composition, including the hydrogel and nanoporous particles, advantageously results in considerably tougher and functional gels which can be triggered by load or stretch signals. Because the particles are preferably porous and hydrophobic, they can be used to dramatically increase the toughness of the gel. Moreover, the surface properties of the particles can be designed such that the material gets activated at desired deformation or impact threshold levels or values.

Moreover, the present hybrid hydrogel and nanoporous particle composition advantageously minimizes reflection and maximizes absorption of the impact energy. With the same energy absorption rate, it is expected that the present gel composition will be at least 40% lighter, at least 67% thinner, and provide 5-10 times more energy absorption per unit weight, in comparison to the prior Trymer® 200L foam of Dow Chemical Co. used in skin composites, by way of example. Additionally, the present composition can be used as implosive based reactive armor incorporating the ultra-tough hydrogel with an implodable internal nanoporous particle structure. The gel benefits from a programmable ultra-large toughness formed by adding the nanoporous particles into the reinforced hydrogels. The programmable gel has a reserve toughness due to the implosion, which can be triggered at various predetermined specific deformation or stress threshold levels or values. Additional advantages and features of the present hydrogel and nanoporous particle composition, and methods, will become apparent from the following description and claims as well as the appended drawings.

### BRIEF DESCRIPTION OF THE DRAWINGS

FIG. 1 is a scanning electron microscope photograph of a hydrogel and a nanoporous particle composition of the present invention;

FIG. 2 is a scanning electron microscope photograph of a silica nanoporous particle employed in the present hydrogel composition;

FIG. 3 is a diagrammatic cross-sectional view, taken along line 3-3 of FIGS. 6 and 7, showing the present hydrogel and nanoporous particle composition applied to rigid outer layers;

FIG. 4 is a diagrammatic cross-sectional view showing the present hydrogel and nanoporous particle composition;

FIG. 5 is a fragmentary cross-sectional view, taken along line 5-5 of FIG. 3, showing one of the nanoporous particles employed in the present hydrogel composition;

FIG. 6 is a perspective view showing an armored land vehicle employing the present hydrogel and nanoporous particle composition with the armored composition layers partially fragmented in a stepped manner;

FIG. 7 is a perspective view showing an armored aircraft vehicle employing the present hydrogel and nanoporous particle composition with the armor layers partially fragmented in a stepped manner;

FIG. 8 is a perspective view showing a wearable helmet employing the present hydrogel and nanoporous particle composition;

FIGS. 9A-D are a series of diagrammatic cross-sectional views showing an energy dissipation function of the present hydrogel and nanoporous particle composition;

FIG. 10 is a graph comparing expected stress versus strain results of the present hydrogel and nanoporous particle composition, conventional DN and SN hydrogels, and porcine cartilage;

FIG. 11 is a graph comparing expected pressure versus volume change results of the present hydrogel and nanoporous particle composition;

FIGS. 12A-C are a series of graphs comparing expected stress versus strain results of the present hydrogel and nanoporous particle composition;

FIG. 13 is a graph comparing expected stress versus volume change results of the present hydrogel and nanoporous particle composition, and a conventional hydrogel;

FIG. 14 is a partially fragmented perspective view showing a vehicular interior seat and head rest employing the present hydrogel and nanoporous particle composition;

FIG. 15 is a cross-sectional view, taken along direction 15-15 of FIG. 14, showing the vehicular interior seat and head rest employing the present hydrogel and nanoporous particle composition;

FIG. 16 is a perspective view showing the present hydrogel and nanoporous particle composition used with a shoulder joint;

FIG. 17 is a cross-sectional view, taken along line 17-17 of FIG. 16, showing the present hydrogel and nanoporous particle composition used with the shoulder joint;

FIG. 18 is an end elevational view, showing the present hydrogel and nanoporous particle composition used with a femur;

FIG. 19 is a cross-sectional view, taken along line 19-19 of FIG. 18, showing the present hydrogel and nanoporous particle composition used with the femur;

FIG. 20 is a diagrammatic side view showing the present hydrogel and nanoporous particle composition used with a hip prosthesis;

FIG. 21 is a stained cross-sectional view, partially enlarged, showing the present hydrogel and nanoporous particle composition used as an organ scaffold;

FIG. 22 is a top elevational view showing the present hydrogel and nanoporous particle composition used as a skull scaffold;

FIG. 23 is a diagrammatic cross-sectional view, like that of FIG. 4, showing an alternate embodiment of the present hydrogel and nanoporous particle composition; and

FIG. 24 is a perspective view showing an alternate embodiment cylindrical nanoporous particle employed in the present composition.

### DETAILED DESCRIPTION

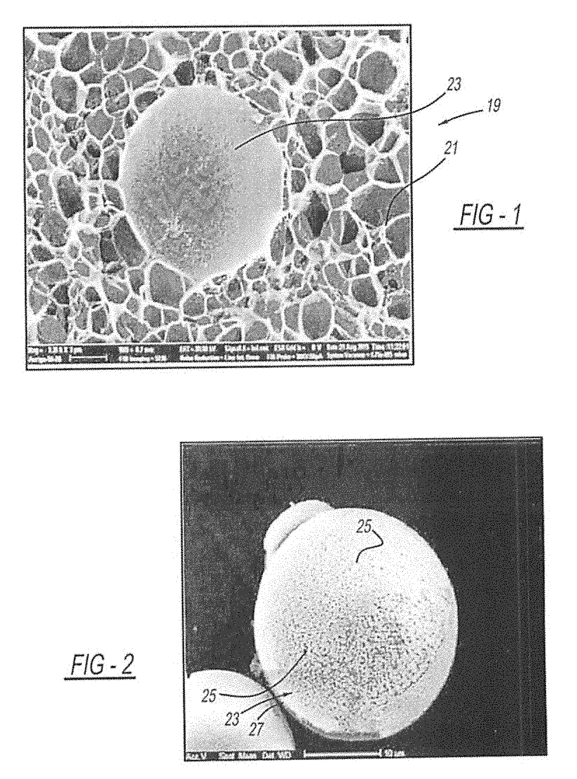

A preferred embodiment of a hybrid composition 19, including a hydrogel 21 and nanoporous particles 23, is shown in FIGS. 1-5. This version employs nanoparticles 23 intermixed within hydrogel 21. Each particle 23 has internal hollow cavities, such as multiple nanopores 25 or channels through an outer wall 27 and/or a central void 29 therein. The cavities contain a gas, such as air, or may alternately be empty, but are nevertheless substantially vacant of and do not contain a liquid when in an ambient or normal uncompressed condition, even when surrounded by hydrogel 21 as is illustrated in FIG. 4. A porous microstructure of hydrogel 21 with an embedded SPO1 nanoporous particle 23 is shown in the enlarged FIG. 1. Adding the SPO1 nanoporous particle into the hydrogel network does not change the pore size. At the edge of the SPO1 nanoporous particle, physical bonds can be directly observed indicating that certain nanoporous particles 23 can be selected to act as a physical cross-linker in hydrogel 21 whose mechanical strength is thereby improved. The physical cross-linking of the SPO1 nanoporous particle relies on entanglement, adhesion, friction and other reinforcing mechanisms to improve the strength of the hydrogel polymer chains similar to those in reinforced rubbers. Alternately, chemical cross-linking may be employed to create covalent bonds between the nanoporous particles and the hydrogel polymer chains. FIG. 2 is an enlargement of FIG. 1 and outer wall 27 of a spherical silica nanoporous particle 23 is hydrophobic so that liquid will be repelled or kept out of nanopores 25 at the ambient condition.

The nanoporous particles 23 each have an extremely large surface area and excellent cost-performance ratio. To precisely control the activation stress level of the reserve toughness of hybrid hydrogel composition 19, the porous structure of the particle should be restrained. Silica is a preferred nanoporous particle material due to its variety in particle morphology, nanoporous structure, and low cost. The nanoporous particle additive frame material may alternately include: (a) ceramics including but not limited to silica gels, zeolites, glass, carbon nanotubes, graphene, active carbon, alumina, or silicon; (b) metals including but not limited to gold, silver, palladium, platinum, copper, copper-based alloys, aluminum-based alloys, magnesium-based alloys, or zinc-based alloys; or (c) polymers including but not limited to cellulose acetate, nitrocellulose, cellulose esters, polyethylene, polypropylene, polysulfone, polyether sulfone, polyacrilonitrile, polyamide, polyimide, polytetrafluoroethylene, polyvinylidene fluoride, polyvinylchloride, covalent organic framework based polymers, covalent triazine framework based polymers, polymers of intrinsic microporosity, crosslinked polymers, or conjugated microporous polymers. The nanoporous structure governs system parameters on reserve toughness and failure strength of the hybrid hydrogel.

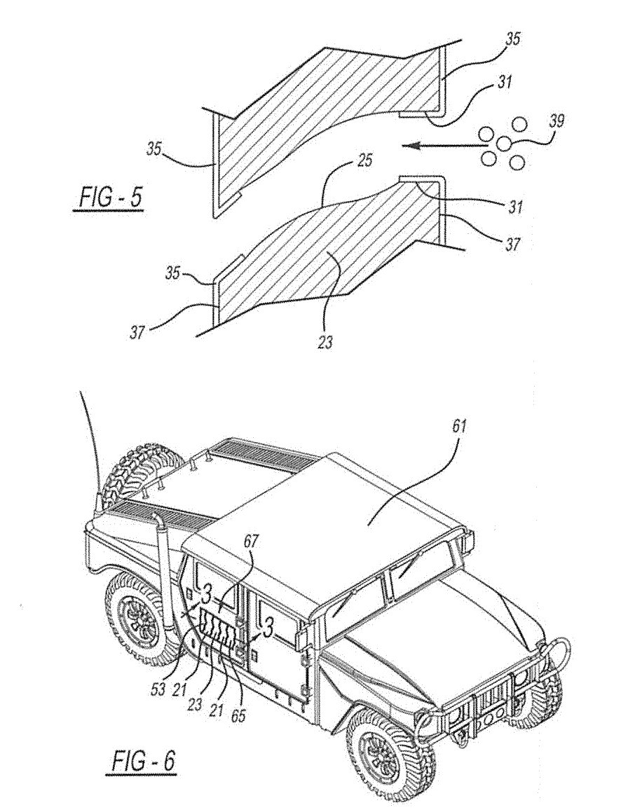

As seen in FIG. 5, to make nanoporous particles 23 stress-field responsive, special surface treatment needs to be applied on the inner surface or open edges 31 of nanopores 25. In nature, the surface of the particle is hydrophilic; when these particles are immersed into a wetting liquid, the nanopores are occupied by the liquid molecules immediately, which prevents energy from being dissipated by such an untreated system. Accordingly, an organic hydrophobic layer or coating 35 can be used to cover the original outer surface 37 which dominates the surface properties of each nanoporous particle 23.

Various methods can be employed to provide a hydrophic coating or layer surface treatment at the pores or cavities of the particles. In one exemplary approach for silica nanoporous particles, the nanoporous particles are vacuum dehydrated at 120° C. for 12 hours and then refluxed in a 2.5% dry toluene solution of chlorotrimethylsilane at 90° C. for 24 hours. Thereafter, they are rinsed with dry toluene then methanol. In another method, the vacuum dehydrated nanoporous particles are refluxed in a 5% dry toluene solution of chlorodimethyloctylsilane at 90° C. for 24 hours. After rinsing in dry toluene then methanol, an encapping treatment is performed. For all these approaches, the nanopore inner surfaces are coated with a mono-layer of hydrophobic silyl groups. This deters the entry of liquid 39 into pores 25 under pressures below the activation level due to a capillary effect.

Since the degree of hydrophobicity of the present nanopores is increased, the excessive solid-liquid interfacial tension can be increased to inhibit the invasion or inflow of liquid molecules. To avoid this issue, surfactants can be used to improve the dispersion of hydrophobic nanoporous particles in solvents. The increased degree of hydrophobicity of the nanoporous particle will make the dispersion more challenging while the use of the surfactant will further reduce the surface tension of the liquid phase. The coupling effect of reduced surface tension and the low solubility of nanoporous particles is considered and circumvented by reducing nanopore size, increasing surface coverage, changing surface reagents, and/or using an end capping technique to increase the degree of hydrophobicity of the nanopores. On the other hand, surfactants with relatively high surface tension or longer chains may be employed to homogenize the solution. Due to the small size of the nanopores, surfactants with longer chains cannot physically enter the nanopores. In addition, ultrasonication can help enhance the particle dispersion in the liquid phase. Pluronic F127 (PF127) is an exemplary surfactant that can be used. The nanopore inner surface is non-wettable to the liquid phase.

In an alternate embodiment shown in FIG. 24, each particle 223 has a substantially cylindrical shape. These are also known as nanotubes. Open ends 224, a central hollow cavity void 229 and nanopores 225 are all hydrophobically coated to repel entry of liquid in the hydrogel in the ambient condition. Irregularly shaped nanoporous particles may alternately be employed.

Hydrogel 21 is a polymeric material containing a liquid such as pure water, salt water, ethanol, oil or the like. The types of monomers, polymeric network configurations, and processing methods will affect the failure strength, toughness and protection efficiency of the hybrid hydrogel. Single network hydrogels, hydrogels with reinforced networks including double network, and interpenetrating network (“IpN”) hydrogels, may be used. Exemplary polymer matrix materials for hydrogel 21 can be classified into three categories, (a) homopolymeric single network, (b) copolymeric, and (c) interpenetrating polymeric networks (of which the double network is a subset). Monomers include but are not limited to: acrylamide, acrylate, acrylic acid, agarose, ampholytes, carboxymethyl cellulose, cellulose derivatives (such as hydroxypropylmethyl cellulose), cyclohexyl methacrylate, dimethylsiloxane, dococyl acrylate, ether, ethylene glycol, ethylene glycol methacrylate, ethylene glycol methyl ether methacrylate, ethyl methacrylate, ethylene oxide, glycolic acid, hyaluronic acid, lactic acid, methyl methacrylate, potassium acrylate, propylene oxide, saccharide, silicone, sodium acrylate, sodium allyl sulfonate, sodium styrene sulfonate, stearyl methacrylate, styrene, triethylenglycol dimethacrylate, urethane, vinyl alcohol, vinyl amine, vinyl phosphonic acid, acrylamido-2-methylpropane sulfonic acid, 2-hydroxyethyl methacrylate, 2-methacrylamidopropyltrimethyl ammonium chloride, (2-(methacryloyl)ethyl) dodecyldimethylammonium bromide, 3,4-ethylenedioxythiophene, 4-t-butyl-2-hydroxycyclohexyl methacrylate, (11-(acryloyloxy)undecyl) trimethylammonium bromide, ε-caprolactone, cis-1,2-bis(2,2-epoxybutanoyloxy)-3,5-cyclohexadine, N-butyl methacrylate, N-isopropyl acrylamide, N-vinyl-2-pyrrolidone, N-iso-propylacrylamide, N,N-dimethylacrylamide, or N,N′-methylenebisacrylamide

More specifically, a first example of a single network hybrid hydrogel composition 19 is set forth as follows. N,N′-methylenebis (acrylamide) (MBAA, 99.0%, Sigma Aldrich Co.) is recrystallized from methanol before use. Acrylamide (AAm, 99%, Sigma Aldrich Co.), potassium persulfate (KPS, 99%, Alfa Aesar) and Pluronic F127 (PEO₉₉—PPO₆₅-PEO₉₉, Sigma Aldrich Co.) are preferred. A preferred nanoporous material is a hydrophobic coated silica SPO1 (SP-120-10, Daiso Corp.).

Hydrogels 21 are synthesized via a one-step sequential free radical polymerization. Silica gel SP01, 1.78 g AAm and 0.038 g surfactant F127DA are mixed in 5 mL water. Then, after bubbling under a nitrogen atmosphere for at least 30 minutes, 0.1 mol % of the initiator potassium persulfate (KPS) with respect to AAm are added into the mixture. The solution is thereafter poured into a mold. The mold is heated at 50° C. for 12 hours in a water bath for polymerization and gelation.

A second example uses a double network hybrid hydrogel composition 19 and is set forth as follows. 2-Acrylamido-2-methylpropanesulfonic acid (AMPS, 98%, Alfa Aesar) and N,N′-methylenebis (acrylamide) (MBAA, 99.0%, Sigma Aldrich Co.) is recrystallized from methanol before use. Acrylamide (AAm, 99%, Sigma Aldrich Co.), potassium persulfate (KPS, 99%, Alfa Aesar) and sodium dodecylbenzenesulfonate (SDBS, 99%, Alfa Aesar) are selected and used. The nanoporous material is a hydrophobic coated, precipitated silica IR01 (Perform-O-Sil 668, Nottingham Corp.).

The DN hydrogel is thereafter synthesized via a two-step sequential free-radical polymerization. In the first step, silica gel IR01 and 0.01M SDBS are added into 1 M AMPS solution and the mixture is sonicated for 5 minutes until fully dispersed. Then 4 mol % MBAA and 0.1 mol % potassium persulfate (KPS) with respect to AMPS is added into the mixture and stirred for 30 minutes in a nitrogen gas atmosphere. Next, the solution is poured into a glass reaction cell sealed by a silicone rubber spacer. The cell is heated at 60° C. for 10 hours in a water bath. After gelation, the PAMPS gel is immersed into a large amount of 2 M AAm aqueous solution containing 0.1 mol % KPS and MBAA for one day. The swollen gel is then heated at 60° C. for 10 hours for polymerization. To remove the residual substances, the PAMPS/PAAm DN gel is thereafter rinsed in water for one week before use. The silica reinforced DN gels should possess excellent compressive properties. When the content of IR01 is 0.25 wt %, the expected fracture stress of IR01 reinforced DN gels significantly increases from 17.3 to 56.9 MPa at a strain of 0.98. Thus, IR01 nanoporous particles are very effective to reinforce the PAMPS/PAAm DN gels.

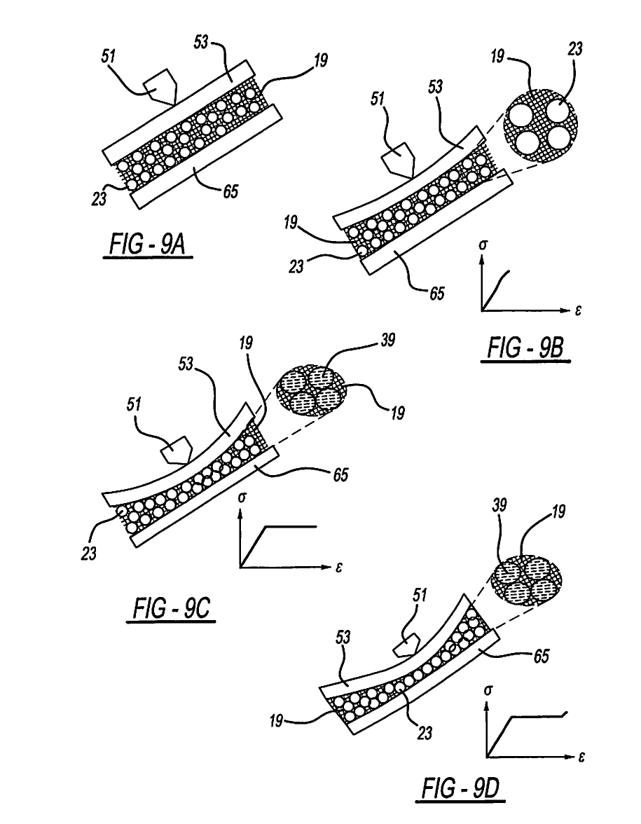

FIGS. 9A-D illustrate the impact penetration process of an external projectile 51 against an implosive reactive, outer armor layer 53 employing the present hybrid composition as a liner sandwiched between two ceramic or metal plates 19. FIG. 9A illustrates the composition before impact; FIG. 9B at initial impact; FIG. 9C when impact energy reaches an implosion trigger or threshold level where implosion takes place; and in FIG. 9D after implosion, where the gel network yields in the last stage which also dissipates the remaining impact energy of the projectile. The proposed implosive based reactive armor incorporates the present ultra-tough hydrogel composition with an implodable internal structure.

The hydrogel composition benefits from the programmable ultra-large toughness formed by adding the nanoporous particles (“NpP”) into the reinforced hydrogels. The method leads to formation of a functional gel with a reserve toughness, which can be triggered at a specific customizable load level or threshold. Upon activation, the nanoporous particles allow liquid infiltration where liquid 39 from hydrogel 19 penetrates into nanopores 25 and dissipates a large amount of energy as excessive solid-liquid interfacial tension and friction. When impact loads are smaller than the activation load or threshold, the composition material will behave as a soft, flexible and resilient layer. For example, the hydrogel and nanoporous particle composition behind armor layer 53 can be programmed or tailored to implode once the impact energy reaches the yield stress of outer steel armor layer 53. This absorbs the energy of the pressure waves before any internal occupant injury occurs. The programming or customization takes place at compounding level through changes in or varying of the morphology, surface properties, types, and load fraction of the nanoporous particles (or any combination thereof).

The “programming,” customization, tailoring, varying or setting of the predetermined threshold value can be structurally changed by varying the pore size, volume and/or quantity in each particle. Such features allow tailoring of the constitutive behavior by adjusting the height and width of the plateau created by each nanoporous particle type in a constitutive curve. For example, the linear opening dimension or diameter of a silica pore is 2-400 nm and more preferably 50-400 nm. The carbon pore dimension or diameter is 2-100 nm and more preferably 2-15 nm.

The threshold setting can be morphologically varied by using different nanoporous particle shapes, for example spheres, nanotubes, disks or other irregular forms, and/or sizes, for example a spherical diameter of 3-100 microns. Furthermore, the surface property threshold setting can be varied by use of different surface modifiers for the inner and/or outer NpP surfaces such as selecting silane groups, mercaptohexadecanoic acids (MHA), 3-(Trimethoxysilyl)propyl methacrylates (TMSPMA), or other hydrophobic coatings depending on the base nanoporous particle material employed. These variables will provide differing toughness or other performance characteristics affecting threshold values.

The NpP to hydrogel loading factor can be alternately or additionally used to vary the composition threshold. For example, an NpP loading fraction greater than 0 wt % to 10 wt % is desired when the NpPs are synthesized into the polymer network in an intermixed manner like FIG. 4. In the sandwich structure of FIG. 23, however, an NpP loading fraction greater than 0 wt % to 50 wt % may be employed, but 20-40 wt % is preferred. Moreover, the chemistry of the composition can be alternately or additionally varied to set the threshold; for example, using ceramics versus metals versus polymers versus elastomers for the NpP material. These variables will provide differing toughness or other performance characteristics affecting threshold values.

Nanopores 25 remain empty in an ambient condition (i.e., in a no load or minimal load situation) when hydrophobic particles 23 are dispersed in the liquid phase. If an external impact loading passes the predetermined threshold value and overcomes an energy barrier associated with the capillary effect of the coated pores, liquid molecules 39 can be forced into and infiltrate the nanopores. When the PAMPS/PAAm SP01 hybrid hydrogel composition is compressed, no fracture should be observed and it should be intact after the loading. The threshold between the elastic and plastic regions is the liquid infiltration pressure of SP01. Referring to FIG. 11, below Pᵢₙ, the constitutive behavior of the present hybrid hydrogel is purely elastic and non-hysteretic as indicated by the first two loading compression cycles. Once Pᵢₙ is reached, liquid infiltration takes place and the material yields. The yield strength and the specific volume change of the hybrid hydrogel are about the same as the Pᵢₙ and Vᵢₙ of SP01, respectively. The liquid infiltration pressure, Pᵢₙ, and the total deformability of the system, Vᵢₙ, are also depicted. Pᵢₙ is proportional to the effective excessive solid-liquid surface tension, while V_{II}, is the total pore volume of particles 23. Hence, the reserve toughness of the hybrid hydrogel is determined by the nanoporous particle. When the nanopores are filled by the liquid molecules, the bulk modulus of the system increases dramatically. Due to the zero liquid outflow of this exemplary nanoporous particle and liquid combination, the yielding is irreversible. However, no permanent damage in the hybrid hydrogel occurs. Therefore, mechanical behavior of flexible, strong and tough hybrid hydrogel system is achievable. Alternately, it is envisioned that liquid infiltration into certain nanoporous particles can be reversible (i.e., the liquid is expelled after impact) so that the hybrid composition can be reused or impacted multiple times without the need for replacement.

In DN gels, extensive bond breakage occurs in the first network in the early stages of tensile deformation. Considerable damage is taking place at the first network even in the small deformation ranges. It is hypothesized that after bondage breakage, the chains of the first network will recoil and form smaller polymer clusters. Necking instability has been observed in some traditional reinforced hydrogels as an unstable competition between the damage-induced softening of the first network and the stiffening of the second network. In the present hybrid hydrogel and nanoporous particle composition, however, the effect of reserve toughness in compression is quite similar to the necking also in tensile. Accordingly, the reserve toughness results from the high amount of energy taken by the particles to allow liquid infiltration into the nanopores or voids.

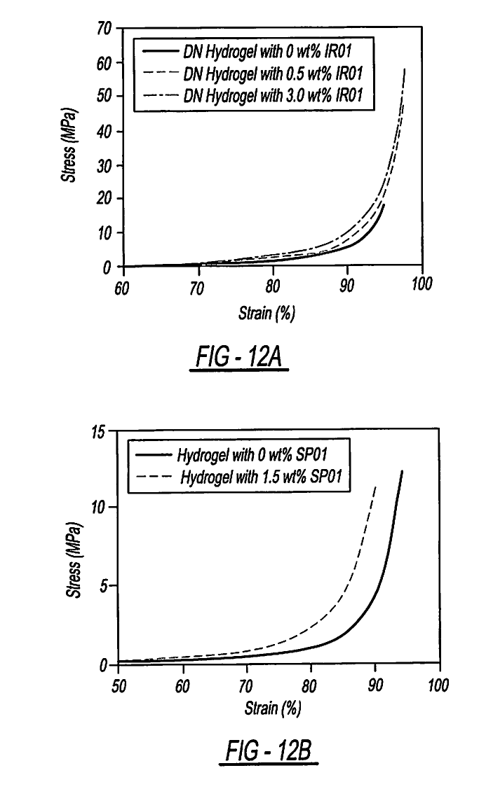

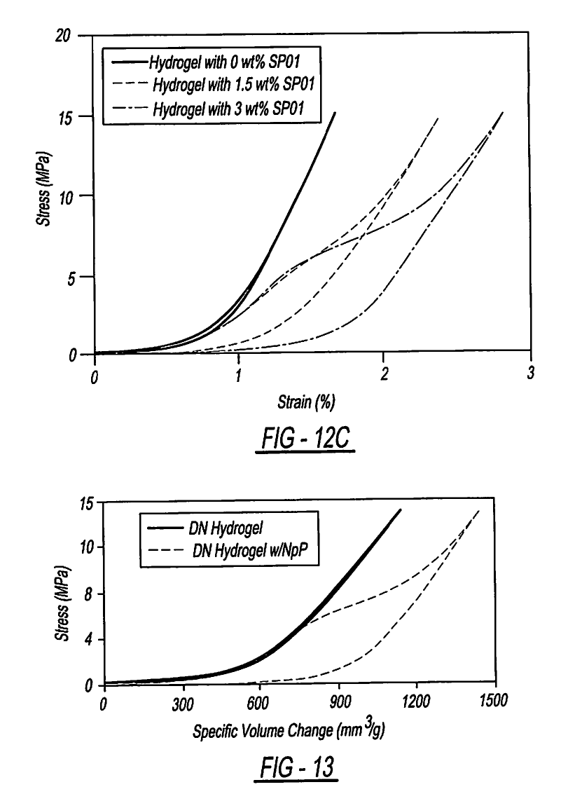

Exemplary confined compressive stress-strain curves are shown in FIG. 13. When an isotropic hydrostatic pressure is applied, the soft hydrogel matter deforms and the system free energy increases as the stored strain energy. The liquid molecules tend to diffuse from the high-energy region (the hydrogel network) to the low-energy region (the small pores or voids in the nanoporous particles). As the PAAm hydrogel without NpP is compressed, since there are no voids in the network, the sorption isotherm curve increases linearly except for the initial concave section; conversely, upon unloading, the curve almost coincides with the loading curve. As the SPO1 NpP filled PAAm hydrogel is compressed, liquid molecules cannot infiltrate into the nanoporous silica initially when the pressure is low, and the sorption isotherm curve increases linearly. But when the pressure reaches 5 MPa, the slope of the sorption isotherm curve decreases suddenly, indicating that the pressure induced liquid infiltration into the nanopores begins. The plateau shows a positive slope due to the pore size distribution. Most of the pores are saturated and the system compressibility decreases rapidly when the pressure reaches 9 MPa. The pressure range of the infiltration plateau is expected to be similar to that of SPO1 in F127 aqueous solution, suggesting that the confined water molecules in nanopores dominate this process. The expected unloading behavior is linear, similar to the F127 solution based nanoporous system. The energy absorption capacity of the present nanoporous particle reinforced hydrogel is expected to be 2 J/g while a conventional hydrogel is expected to be 0 J/g. By controlling the nanopore size, the surface condition and the liquid types of the present composition, the energy absorption triggering pressure or threshold can be adjusted or customized in the range of 0.5 MPa to 40 MPa or even higher. Consequently, it is expected that the maximum energy absorption capacity of at least 100 J/g can be achieved.

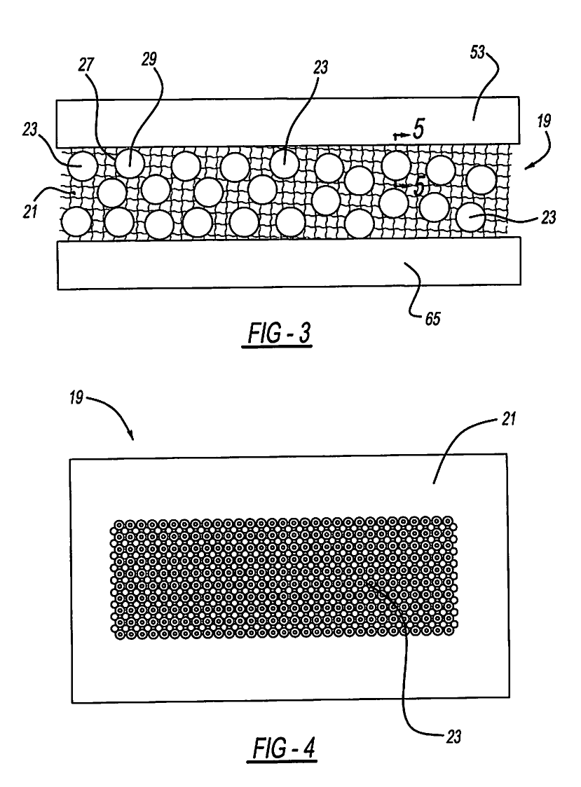

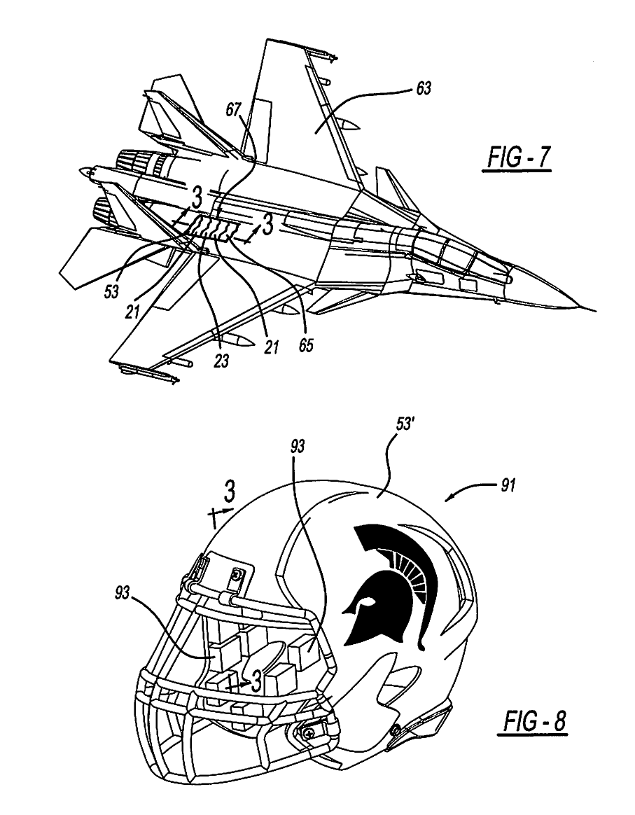

Reference should now be made to FIGS. 3, and 6-9. The present hybrid hydrogel and nanoporous particle composition 19 provides an ultra-fast energy dissipation mechanism for skin or shell composite assemblies such as for use in military vehicular structures to mitigate high strain rate deformation and multi-frequency blast waves. It is also ideally suited for use in wearable body armor such as vests and helmets. The addition of nanoporous particle 23 into the aqueous-based polymer network of hydrogel 21 allows engineering of a dynamic response of the material to external loads, rather than objectively relying on the visco-elastic properties of conventional rubber-like foams. The nanoscale pores in the particles respond to external loading in a few microseconds, which is much shorter than a typical explosive blast wave front rising time, which is hundreds of microseconds. With this, the reserve toughness and liquid infiltration into the NpNs can be activated before the blast wave reaches its peak value. In addition, the liquid infiltration pressure (activation pressure of the reserve toughness) can be varied and controlled precisely by the previously discussed system parameters and characteristics in a wide range. During an impact scenario, the core can be activated at the exact desired stress level or threshold value. Since the infiltration takes place at constant stress, the large volume changes associated with the absorbed liquid leads to a high intake and dissipation of energy.

An armored wheeled automotive vehicle 61, such as a truck, personnel carrier, tank or amphibious combat vehicle, an aircraft vehicle 63, or a marine vehicle such as a ship or submarine, employs the present hybrid hydrogel and nanoporous particle composition 19. Such vehicles include exterior armored metallic or ceramic layer 53 and an interior substrate metallic layer 65, between which is hybrid hydrogel and nanoporous particle composition 19. The composition may be of the intermixed type of FIGS. 3 and 4, or of a three (or more) layered sandwich such as that shown in FIG. 23. The tailorable imploding hydrogel composition 19 acts as an inner liner of reactive armor assembly 67, which is exceptionally dissipative, resistive to blast waves, considerably lighter and requires metal layers 53 and 65 only to prevent penetration and not for their impact energy dissipation. Pound for pound, the proposed implosive reactive armor assembly is stronger than traditional non-explosive reactive armor (“NxRA”).

The impact energy will be dissipated mainly through the implosion of hydrogel 21, and partly due to local bending of armored metal shell 53, deformation of projectile 51, and the final yielding of the nanoporous particle polymer matrix. Extreme temperature impact, due to impact of explosive projectile 51 to armor assembly 67 is advantageously dissipated. Explosive projectiles 51 are very effective due to the resulting thermal wave, which softens the NxRA traditional exterior shield. However, the present assembly 67 provides considerable advantages against thermal waves. Upon impact, it is expected that the mechanical blast wave reaches gel 21 first. The counteracting process is similar to ambient temperature impact, although the subsequent to risk of penetration is considerably higher. Then, the thermal wave reaches the gel after implosion, and hits the liquid environment of the gel structure. Due to the specific heat of the liquid media and the encapsulated environment of the gel, a considerable amount of energy will be absorbed in liquid evaporation. A consequent volume change also takes energy from the armor due to an expansion phase which requires destruction of nanoporous particle polymer matrix 23 and the encapsulated shell. Moreover, the process slows down the temperature increasing rate on outer metal shell or layer 53 which helps the shell to keep its mechanical rigidity for a considerably longer time below a critical failure point.

Reference should now be made to FIGS. 3 and 8. Wearable armor such as a sports, construction or military helmet, body armor vest, football shoulder pads, motorcycle jacket elbow or shoulder pads, or the like, advantageously employ the present hybrid hydrogel and nanoporous particle composition 19 to absorb impacts. More specifically, an exemplary American football helmet 91 includes multiple pads 93, a rigid polymeric outer shell 53′, a soft polymeric skin, and an optional foam or fabric inner liner layer 65 that is more comfortable for occupant skin contact. Hybrid hydrogel and nanoporous particle composition 19 is adhesively sandwiched between shell 53′ and liner 65 to define each pad 93. Alternately, hybrid composition 19 may be removeably attached to shell 53′ and/or liner layer 65 via hook and loop fasteners, snaps, or the like, for replacement or cleaning.

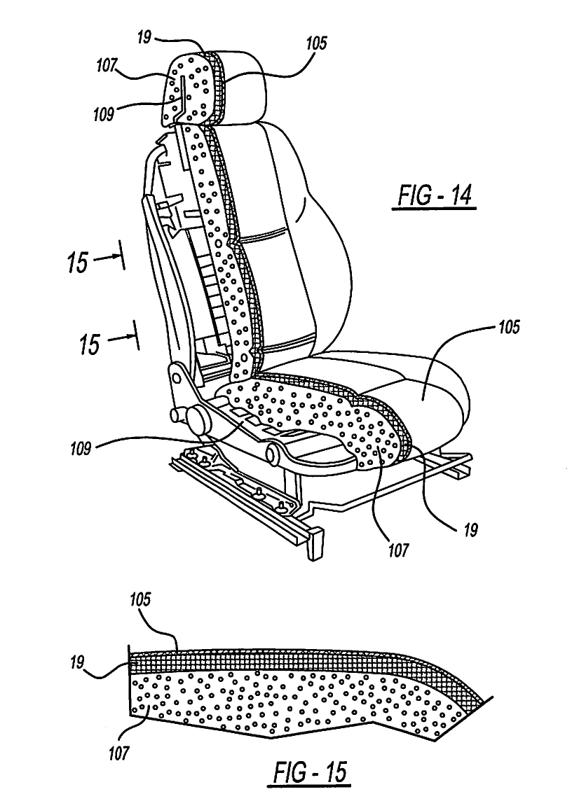

FIGS. 14 and 15 illustrate a vehicular seat assembly, here an automotive wheeled vehicular seat 101 and headrest 103. The present hybrid hydrogel and nanoporous particle composition 19 is sandwiched between an exterior layer 105 of fabric or leather, against which an occupant contacts, and an interior hidden soft foam cushion layer 107. A metallic seat frame or substrate 109 internally supports the foam seat and headrest. The present hybrid hydrogel and nanoporous particle composition 19 is also ideally suited for use in other vehicle interior trim panels, between an external skin layer of fabric, leather or vinyl, and an internal hidden layer of a rigid polymeric substrate, a soft foam and/or a metallic bracket. Such exemplary interior trim panels include instrument panels, dash pad panels, door trim panels, knee bolsters, pillar garnish moldings, sun visors, headliners, steering wheel airbag covers and the like. The seats and headrests are considered as examples of “interior trim” for purposes of this application. The hybrid hydrogel and nanoporous particle composition absorb occupant impacts in a collision situation for both the vehicular interior trim and helmet configurations. In all of the preceding application examples, the rigid or foam layer 53, 53′ and 107 have a thickness dimension less than half of a length dimension and less than half of a width dimension; for example, they may be enlarged sheets of relatively thin material.

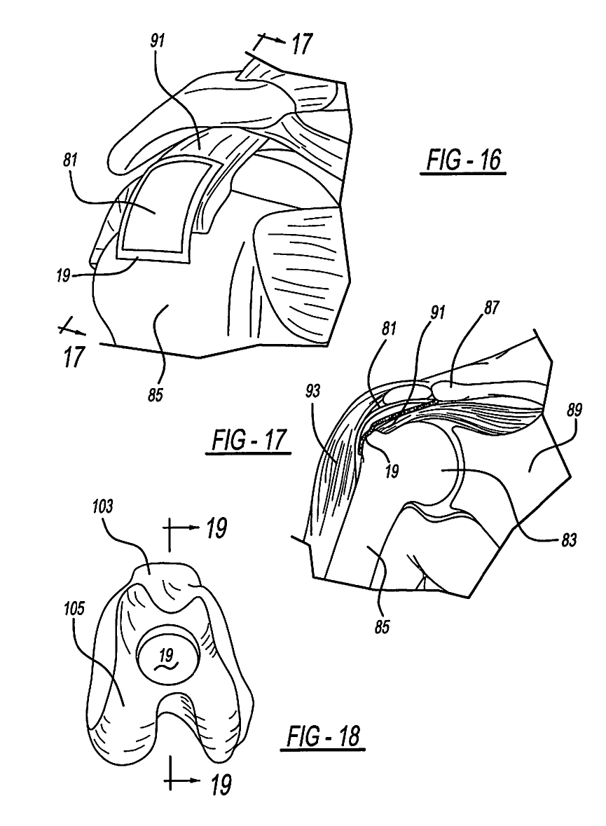

FIGS. 16-22 illustrate biomedical uses for the present hybrid hydrogel and nanoporous particle composition 19. In exemplary FIGS. 16 and 17, composition 19 is part of a tendon repair kit where composition 19 is employed as a secondary supportive and load-bearing layer between a flexible polymeric repair patch 81 and a head or ball 83 of a humeral or arm bone 85 adjacent a collar or clavicle bone 87 and socket or glenoid bone 89. Adhesive cement, staples and/or sutures are used to directly attach the flexible and generally rectangularly shaped composition 19 to one or more of the bones, to a rotator cuff tendon 91, to patch 81 and/or to a deltoid muscle 93. The hydrogel and nanoporous particle composition advantageously prevents or deters excessive loading to the repair patch 81 when the patch is undergoing tension during tendon and muscle movement. Composition 19 will translate the tension to a compressible load passed from the repair patch to the bone seat.

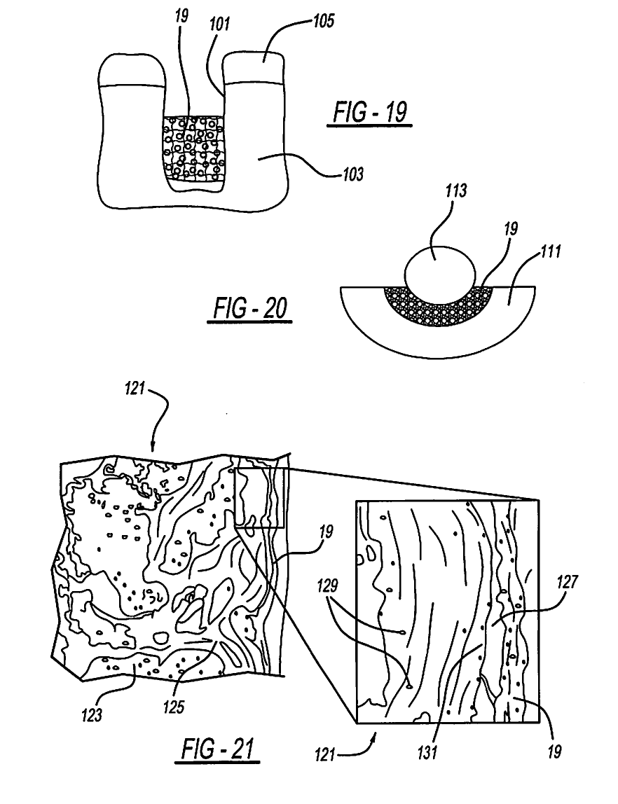

FIGS. 18 and 19 illustrate hybrid DN hydrogel and nanoporous particle composition 19 as a plug to fill an osteochondral defect or gap in a groove 101 of a femur bone 103 at a patellofemoral joint 105. Composition 19 acts as a replacement cartilage. During healing, composition 19 can be tailored or programmed to vary the fluid infiltration threshold in order to dissipate shocks or impacts during bone-to-bone jarring use. For example, the threshold or trigger level can be set to a critical load limit or failure point of an underlying cartilage regeneration scaffold. Composition 19 is adhesively bonded, sutured or stapled directly to the scaffold or bone.

As can be observed in FIG. 20, a hybrid DN version of hydrogel and nanoporous particle composition 19 is coupled inside a concave receptacle of a hip bone-mounted acetabular cup 111, such as one made with a UHMWPE liner. A metallic ball 113, joined to a femoral bone, contacts and slides against an exterior surface of composition 19 located between the ball and the cup. Composition 19 is designed to mimic mechanical behavior of articular cartilage and is expected to have superior durability and service life through its low friction coefficient, and its ability to absorb sudden and excessive impact shook loads. Thus, the NpN impact absorption, through liquid filtration after a predetermined threshold is reached, is ideally suited for all bone and joint prosthetics, while providing a biologically-friendly material.

FIG. 21 shows hybrid DN hydrogel and nanoporous particle composition 19 implanted into subcutaneous tissue 121 of a mammal, such as a human or animal. This is well suited for human knee cartilage regeneration scaffolding. G₄RGDY-modified alginate 123, newly formed bone 125, osteoblasts 127, osteocytes 129 and lamellae 131 are shown in the figure. Composition 19 advantageously provides tailorable or customizable constitutive behavior to match specific behavior of an organ, such as by changing the nanopore size and/or quantity, and/or by varying the particle shape or material selection in the composition. The elasticity, compressibility, viscoelastic behavior, tensile strength, failure strain, gelling conditions, swelling and degradation characteristics of the hydrogel and nanoporous particle composition create synergistic benefits for a tissue scaffold.

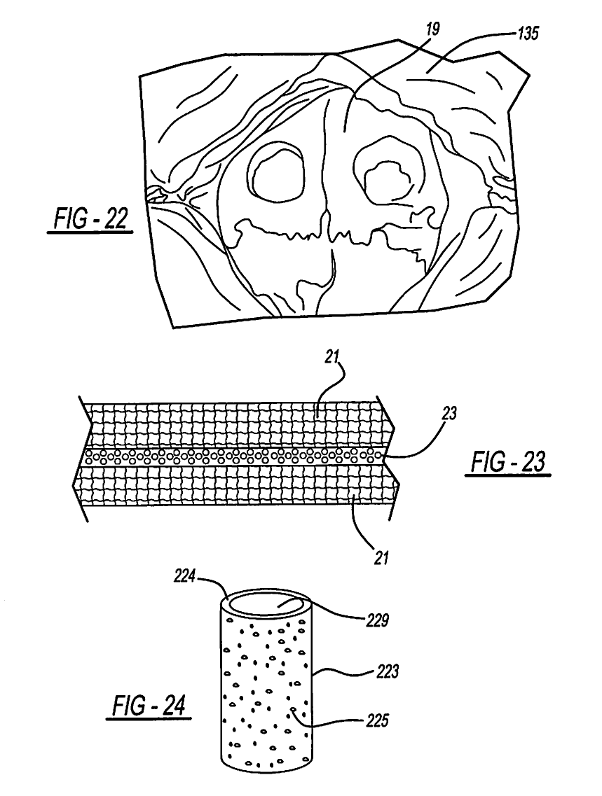

Referring to FIG. 22, a hybrid DN version of hydrogel and nanoporous particle composition 19 is used as an outer cover or shell to protect an underlying tissue regeneration scaffold against undesired shock or impact loads. Composition 19 and the scaffold are adhesively bonded, stapled and/or sutured to a skull bone 135. The tailoring or customizing of the shapes, sizes and/or material characteristics of the composition set the pre-determined liquid infiltration threshold value at or near a critical load resistance value of the skull. Moreover, the present composition 19 is ideally suited to induce bone formation and be biocompatible without brittleness for such cranioplasty procedures.

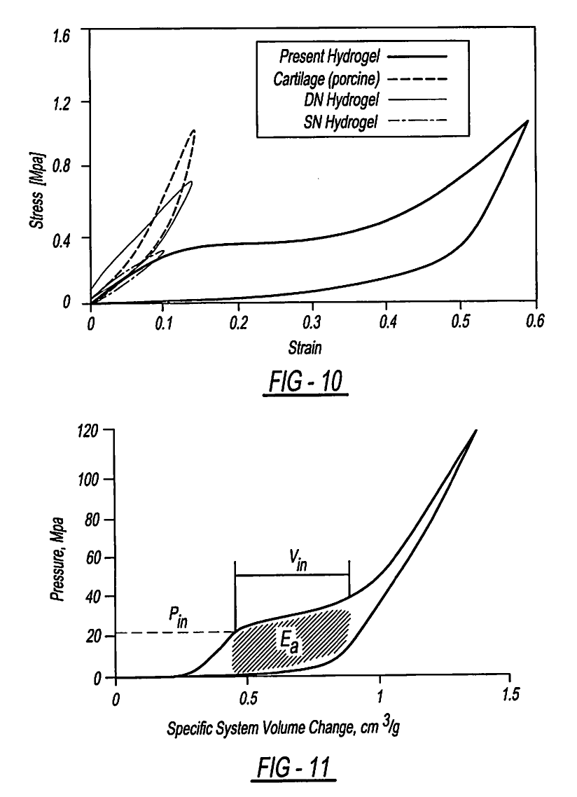

With regard to all of these biomedical uses, the present hybrid composition advantageously minimizes a loss of flexibility. The stiffness of a nanoporous particle is lower than an equivalent solid particle due to the presence of voids. Therefore, the present hybrid hydrogel and nanoporous particle composition are considerably softer than traditional reinforced hydrogels. Furthermore, the previously discussed programmable added or reserve toughness programmed into the material by adjusting the structure, morphology, surface properties and/or loading fractions of the nanoporous particles is advantageous especially in the preceding biomedical uses. FIG. 10 shows an expected comparison of traditional DN and SN reinforced hydrogels versus porcine cartilage versus the present hydrogel and nanoporous particle composition, under compression. The toughness added to the hydrogel by the nanoporous particles (so-called “reserve toughness”) is independent of the interaction between the porous particles and the polymer chains.

Moreover, the present composition enhances failure strength and toughness. The reinforcement in the present hybrid hydrogel composition is the result of two independent mechanisms: (i) strong interaction between the surface of nanoporous particle and polymer chains due to extremely large specific surface area of nanoporous particle for functionalized surfaces; and (ii) the infiltration process at specific stress level. These two independent mechanisms make the present new class of strong and tough hybrid hydrogel achievable for biomedical use. The reserve toughness prominently changes this new type of hybrid gel system into an energy absorber system with high capacity. Furthermore, for all of the disclosed uses, the porous structure can resist heat transfer. In addition, the phase transformation of the confined liquid at certain environment condition (pressure and temperature) can mitigate the transient heat diffusion.

A method of manufacturing the hybrid composition with the vehicular or wearable armor or interior trim is as follows. First, the hydrogel and nanoporous particle is intermixed and prepared into the composition 19 as discussed in the examples hereinabove. Second, either the outer layer or less preferably the inner layer, such as helmet shell 53′, armor plate 53 or seat foam 107, is placed against an inner surface of a rigid mold. Alternately, such a layer can serve in place of a mold if it is rigid enough and has integral upstanding walls surrounding the area of interest or temporary upstanding walls can be sealingly placed against the layer. Third, the intermixed composition is heated to approximately 50-60° C. Fourth, the intermixed composition is poured directly against the mold, outer layer or inner layer. Either the natural adhesive properties of the hydrogel will attach to the outer layer, or an intermediate adhesive film or coating can optionally be applied to the outer layer before the pouring, depending on the specific layer and hydrogel materials used.

Fifth, the optional opposite layer, such as metal substrate 65, helmet interior pad liner 65, or fabric 105, or alternately another mold half, is placed onto the hybrid composition opposite the outer layer or initial mold half before it cures and, optionally, prior to the pouring step. An optional adhesive layer or coating can be placed therebetween if necessary. Sixth, the hybrid composition and optionally, adhesive, are cured at room temperature to create a completed shell and composition sandwich assembly.

FIG. 23 illustrates a sandwich construction alternate embodiment to create the present hydrogel and nanoporous particle composition 19, In this embodiment, one layer of hydrogel 21, without nanoporous particles therein, is compounded, heated, poured into a mold and cured to define a pre-formed layer. Thereafter, dry powdered nanoporous particles 23 are sprayed, coated or otherwise placed on top of the first hydrogel layer. Next, another preformed layer of hydrogel 21, also without particles therein, is placed on top of the intermediate layer of nanoporous particles 23. The intermediate layer is approximately 100 micron-3 mm thick and each of the hydrogel layers are approximately 3-10 mm thick. A flexible, outer polymeric skin layer may thereafter encapsulate the three (or more) layer assembly. The innermost surfaces of hydrogel 21 are punctured if the impact threshold is exceeded to transmit the liquid therein into the nanopores or voids of particles 23.

FIGS. 12A-C illustrate an influence of nanoporous particle loading fraction on the failure strength of the present intermixed hybrid hydrogel and nanoporous particle compositions. FIG. 12A shows a PAMPS/PAAm DN hydrogel with IR01. FIG. 12B shows a PAAm SN hydrogel with SP-01 and FIG. 12C shows a reserved toughness versus nanoporous particle loading fraction. This is in a uniaxial compressive mode. Improvement of the failure strength of the hybrid composition is expected with an increased amount of IR01. But, additional SPO1 is expected to slightly reduce failure strength of the SN hydrogel and nanoporous particle composition. Thus, failure strength of the present composition is believed to be sensitive to the nanoporous particle size and shape.

While various embodiments have been disclosed herein, it should be appreciated that other variations may exist. For example, the present hydrogel and nanoporous particle composition can be used for other biomedical applications such as wound dressings and the like. The present hydrogel and nanoporous particle composition may alternately be used to surround or be a flexible substrate for electronic circuits or sensors coupled thereto. Any and all of the previously disclosed features may be mixed and matched with any or all of the other embodiments. Moreover, all of the following claims can be multiply dependent on each other in any combination. The above description is merely exemplary in nature and, thus, variations that do not depart from the gist of the invention are intended to fall within the spirit and scope of the present invention.

The invention claimed is:

1. A composition comprising a hydrogel and particles, each of the particles including a hollow nanopore that is vacant of a liquid when in an ambient state.

2. The composition of claim 1, further comprising a rigid external layer attached to the hydrogel, the rigid layer having a thickness dimension less than half of a length dimension and a width dimension.

3. The composition of claim 2, wherein the rigid external layer is a helmet shell, the hydrogel and nanoporous particles absorbing an impact on the shell.

4. The composition of claim 2, wherein the rigid external layer is metallic or ceramic armor of a vehicle, the hydrogel and nanoporous particles absorbing an impact on the armor.

5. The composition of claim 2, wherein the rigid external layer is armor, the hydrogel acting as a buffer against thermal shock of a hot projectile.

6. The composition of claim 1, wherein:
the nanoporous particles are vacant of the liquid and the hydrogel operably absorbs an initial impact; and
the liquid enters the nanopores if the impact is greater than a threshold value such that liquid infiltration into the nanopore of the particles assists in absorbing the greater impact.

7. The composition of claim 1, further comprising a hydrophobic layer on the particles to deter the liquid from entering the nanopore.

8. The composition of claim 1, wherein:
the particles have a substantially spherical shape;
there are multiples of the nanopores in each of the particles;
each of the particles has a linear opening dimension of 2-400 nm; and
each of the particles has a linear cross-sectional dimension of 3-100 microns.

9. The composition of claim 1, wherein:
the particles have a substantially cylindrical shape;
there are multiples of the nanopores in each of the particles;
each of the particles has a linear opening dimension of 2-400 nm; and
each of the particles has a linear cross-sectional dimension of 3-100 microns.

10. The composition of claim 1, wherein the hydrogel includes at least two polymeric hydrogel sublayers with a powder or coating layer of the nanoporous particles is sandwiched therebetween.

11. The composition of claim 1, wherein:
the particles are an additive intermixed with the hydrogel;
the particles serve as cross-linkers by interacting with polymeric chains of the hydrogel so as to strengthen the hydrogel polymer chains; and
the nanopores are sized to increase an energy absorption capacity and toughness of the hydrogel.

12. The composition of claim 1, further comprising a biological member comprising: (a) a tendon, (b) a muscle, (c) a tissue, (d) an organ, or (e) a bone, attached to the hydrogel by adhesive, a suture or staple, the liquid entering the nanoporous particles in an impact situation.

13. A composition comprising:
(a) a hydrogel including a liquid;
(b) particles contacting the hydrogel;
(c) a layer of a material different than the hydrogel and the particles, the layer having at least one dimension larger than the hydrogel;
(d) an initial impact force against the layer being at least partially absorbed by the hydrogel; and
(e) a greater impact force against the layer causing liquid from the hydrogel to enter at least some of the particles, wherein the particles at least partially absorb the greater impact force.

14. The composition of claim 13, wherein the layer is a rigid and external helmet shell.

15. The composition of claim 13, wherein the layer is metallic armor.

16. The composition of claim 13, wherein the layer is part of vehicular interior trim.

17. The composition of claim 13, wherein the layer further comprises a biological member comprising: (a) a tendon, (b) a muscle, (c) a tissue, (d) an organ, or (e) a bone, attached to the hydrogel by adhesive, a suture or staple, the liquid entering the particles in an impact situation.

18. The composition of claim 13 wherein:
the particles are an additive intermixed with the hydrogel;
the particles serve as cross-linkers by interacting with polymeric chains of the hydrogel so as to strengthen the hydrogel polymer chains; and
the particles include nanopores sized to increase an energy absorption capacity and toughness of the hydrogel.

19. A method of manufacturing an energy absorbing system, the method comprising:
(a) applying a hydrophobic material onto nanoporous particles, each of the nanoporous particles having a cross-sectional dimension of 3-100 microns;
(b) placing the nanoporous particles in contact with a hydrogel;
(c) deterring liquid from entering at least a majority of nanopores of the nanoporous particles in an ambient condition, each of the majority of the nanopores having a linear opening dimension of 2-400 nm; and
(d) attaching the hydrogel to a surface made of a material different from the hydrogel.

20. The method of claim 19, further comprising pouring the hydrogel in a liquid state against the surface, which is rigid, the surface serving as a mold section to define a shape of the hydrogel after curing.

21. The method of claim 19, further comprising selecting the nanoporous particles of a desired shape and nanopore size to allow the liquid of the hydrogel to enter the majority of the nanopores when an impact force is present against the surface.

22. The method of claim 19, further comprising sandwiching the nanoporous particles between sub-layers of the hydrogel.

23. The method of claim 19, further comprising customizing a composition of the nanoporous particles and the hydrogel by varying at least one characteristic of the composition during its manufacture to vary a threshold where the liquid can enter the majority of the nanopores.
